(19) United States
(12) Patent Application Publication
(10) Pub. No.: US 2006/0282006 A1
(43) Pub. Date: Dec. 14, 2006

(54) Body fat scale having transparent electrodes

(76) Inventor: Steven Petrucelli, Cranbury, NJ (US)

Correspondence Address: PLEVY & HOWARD, P.C., P.O. BOX 226, FORT WASHINGTON, PA, 19034, US

(21) Appl. No.: 11/507,836

(22) Filed: Aug. 22, 2006

Related U.S. Application Data
(63) Continuation of application No. 10/714,570, filed on Nov. 14, 2003, now abandoned.
(60) Provisional application No. 60/426,452, filed on Nov. 14, 2002.

(51) Int. Cl. A61B 5/05

(52) U.S. Cl. 600/547

(57) ABSTRACT

A body composition or body fat scale having a rigid, visible light transmissive platform for supporting a user standing on the scale, a plurality of support assemblies for supporting the platform above a surface, the support assemblies providing at least one sensor for measuring a weight of the user; at least two visible light transmissive, conductive electrodes disposed over the top surface of the platform, for contacting portions of the user's body to provide signal information about the user's body, which is used for measuring the user's body fat or body composition.

This application claims the benefit under 35 USC 119(e) of U.S. Provisional Patent Application Ser. No. 60/426,452, filed Nov. 14, 2002, the entirety of which is incorporated herein by reference.

### FIELD OF INVENTION

The present invention relates to measurement devices in general and more particularly to an apparatus for determining body fat of a biological organism.

### BACKGROUND

There exist in the prior art numerous methods and apparatus for measuring or determining body impedance and body composition (i.e. body fat). P For instance, U.S. Pat. No. 4,144,763 discloses a method of measuring body fat using Boyle's law. U.S. Pat. No. 4,831,526 discloses a system where a fat-to-lean ratio is measured by having a subject stand on a platform and raise his or her heels and then allowing the weight to fall near a transducer to produce a force. The vibrations of the subject cause a data peak to be produced and measured by a computer. A technique for measuring body fat by immersing the subject in a liquid is disclosed in U.S. Pat. No. 5,052,405. U.S. Pat. No. 5,105,825 teaches a method of measuring body fat by transferring controlled volumes of gas between two chambers and measuring pressure while U.S. Pat. No. 5,335,667 measures body composition using bioelectric impedance measurements.

U.S. Pat. No. 5,415,176 issued on May 16, 1999 entitled APPARATUS FOR MEASURING BODY FAT, to Sato et al. discloses a method of determining body impedance using two pairs of electrodes placed at the toes and heels of a person, applying a constant current to the toe electrodes, measuring the voltage at the heel electrodes, and calculating the impedance as the ratio of the measured voltage over the constant current. The body fat is then calculated from the body impedance.

U.S. Pat. No. 6,292,690 issued to Petrucelli et al. discloses an apparatus and method for determining body fat which uses toe and heel electrodes as drive and sense electrodes and applies a constant current source to the drive electrodes to sense an output signal at the heel electrodes.

The above-described body fat scales often have raised (or depressed) portions on the top surface of a platform or housing corresponding to the electrode areas that must contact the associated foot portions to enable current to be passed through the body to determine the associated impedance and ultimately body composition. These electrode areas are often formed of a cold metal that is uncomfortable to the foot of a user. The depressed or raised areas also result in an uneven top surface that may cause further discomfort. A conductive covering may be applied that indicates to a user where to place one's feet on the base while also eliminating direct contact with the cold metal electrodes. However, such cover further raises the electrode areas on which a user is to stand, increasing the unevenness of the top surface and possibly causing additional user discomfort. Still further, such conductive covering is prone to wear, and may result in a tarnished or tattered appearance over time. Alternative approaches are desired.

### SUMMARY

An apparatus for measuring body composition comprises a rigid, light transmissive base or platform having a top surface and a bottom surface. The bottom surface rests upon a plurality of support assemblies having at least one sensor for measuring the weight supported by the assemblies. On the top surface of the light transmissive platform is disposed a plurality of light transmissive electrodes for contacting with corresponding portions of the feet of a user standing on the platform. An alternating signal is applied to the body via at least two of the light transmissive electrodes and an output signal is sensed at at least two of the light transmissive electrodes. The light transmissive electrodes are uniformly disposed on the top surface of the platform. In an exemplary embodiment, the light transmissive electrodes in aggregate occupy a majority of the area of the top surface of the platform. Each of the light transmissive electrodes are electrically separated from one another on the top surface. In a preferred embodiment, the light transmissive electrodes are formed of Indium Tin Oxide (ITO) material and uniformly applied to portions of the top surface of the platform and define quadrants associated with each of the electrode areas. Each of the light transmissive electrodes is coupled to a contact on the bottom surface of the platform for communicating signal information to a processor for measuring body impedance and determining body composition. Each light transmissive electrode may be either directly coupled to a contact on the bottom surface of the platform, or may be indirectly coupled (e.g. resistively or capacitively coupled) via the medium of the platform, for example. Alternatively, contacts disposed on inner surface of the upper housing are positioned so as to be in direct (or indirect, e.g. resistively or capacitively coupled) electrical communication with respective light transmissive electrode areas for communicating signal information to the processor. A display assembly operatively coupled to the processor displays the measured body weight and/or body composition.

A light transmissive electrode operable as a switch may be disposed on an otherwise exposed region of the light transmissive base or platform for activating or deactivating the apparatus.

An apparatus for analyzing human body composition based on a bioelectrical impedance method, comprises a light transmissive base or platform, a plurality of light transmissive electrodes disposed on the top surface of the platform for contacting with an upper or toe portion of a right foot, a lower or heel portion of the right foot, an upper or toe portion of a left foot, and a lower or heel portion of the left foot; contacts disposed on a portion of the top surface or bottom surface of the platform and in electrical communication with the light transmissive electrodes for providing signal communication to a processor for measuring body impedance.

An apparatus for analyzing human body composition based on a bioelectrical impedance method, comprises a light transmissive base or platform, a first light transmissive electrode disposed on the top surface of the platform for contacting with a left foot of a user, and a second light transmissive electrode disposed on the top surface of the platform for contacting with a right foot of the user; first and second contacts disposed on the top or bottom surface of the platform and in electrical communication with the first and second light transmissive electrodes, respectively provide signal communication to a processor for measuring body impedance. Support assemblies disposed on the bottom surface of the platform containing at least one sensor enable weight calculation for determining a user's body weight.

A circuit for measuring body impedance comprises a voltage source; a current source; a first pair of light transmissive electrodes are disposed on a top surface of a light transmissive base or platform for receiving one portion of the body for applying the current source to the body; a second pair of light transmissive electrodes are disposed on the top surface of the platform to receive another portion of the body for sensing a voltage therebetween; and a processor for measuring body impedance and determining body composition based on the sensed voltage signal in response to the applied current source.

### BRIEF DESCRIPTION OF THE DRAWINGS

FIG. 1A is a top perspective view of an apparatus for measuring body impedance or body composition according to an embodiment of the present invention.

FIG. 1B is a partial cross sectional view taken along A-A of FIG. 1A.

FIG. 2 is a top view of an apparatus for measuring body impedance or body composition according to an alternative embodiment of the present invention.

FIG. 3 illustrates a schematic circuit for measuring body resistance or impedance.

FIG. 4 is a block diagram of the major functional components of the body impedance or body composition measuring apparatus.

FIG. 5 is a perspective view of an alternative embodiment of the scale of the present invention wherein contacts are formed on an interior surface of the upper housing.

FIG. 6 is a plan view of the bottom surface of the platform of the body fat scale of FIG. 5 illustrating the inner surface of the upper housing containing contacts in electrical communication with each of the light transmissive electrodes.

FIG. 7 is an exemplary illustration of the base platform having disposed on its top surface light transmissive ITO electrode materials formed into quadrants.

### DETAILED DESCRIPTION

Body impedance analysis (BIA) is used for estimating body composition. This theory is based on the volume conductor theory, which suggests that the volume of a conductor can be determined by its impedance to current flow. The impedance of a conductor is proportional to its length and is inversely proportional to its cross-sectional area. Thus, the impedance Z of a conductor may be characterized by the equation Z=w*(L/A) where w is the specific impedance of the conductor, L is the length of the conductor, and A is the cross-sectional area of the conductor. Similarly, the volume V of a conductor can be calculated by measuring the length L and the specific impedance w of the conductor (V=w*(L*L/Z). Lean Body Mass (LBM), defined as total body mass less fat body mass, may be estimated since it is known that LBM is a function of total body weight. Once LBM is known, the percentage of body fat (%BF) can be determined according to the equation %BF=100*(Wt−LBM)/Wt where %BF is the percent body fat, LBM is the lean body mass, and Wt is total body weight.

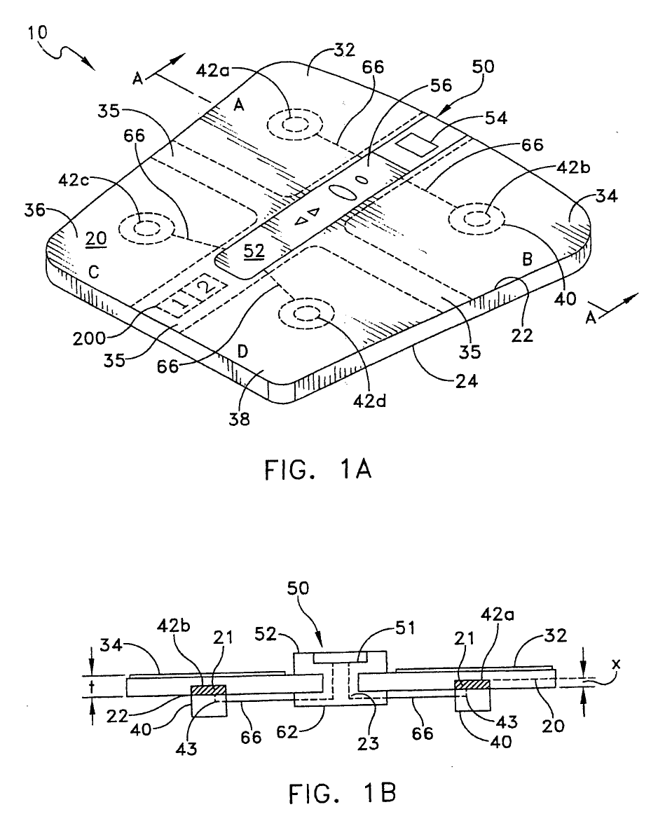

FIG. 1A shows an exemplary embodiment of a body composition or body fat scale 10 according to the present invention. The scale 10 comprises a base or platform 20 having a substantially planar top surface 22 and a bottom surface 24. In the exemplary embodiment, the platform 20 is formed of a rigid, visible light transmissive material including, without limitation, at least a translucent or substantially transparent material, such as a glass or plastic, capable of receiving the weight of a user. A plurality of supports 40 are typically attached to the bottom surface 24 of the platform 20 in a symmetrical or evenly-spaced arrangement so as to support the platform 20 above a ground, floor, table, or like surface in a stable and safe manner. In the shown embodiment, the platform 20 has a square or rectangular configuration, however, platforms having circular, elliptical, oval, triangular, octagonal, or other desired configurations, are also contemplated. Each support 40 contains a sensor, such as a piezoresistive element or load cell, that changes an electrical parameter (e.g. resistance) in response to a weight applied to the platform 20.

As shown in FIGS. 1A and lB, electrical conductors 66 or other electrical connection means, extend beneath the bottom surface 24 of the platform 20 from the supports 40 and through a lower housing 62 of a display assembly 50, to electrically connect each of the sensors to weight calculating and body impedance and composition measuring circuitry 51 contained within an upper housing 52 of the display assembly 50. The portions of the conductors 66 extending between each support 40 and the lower housing 62 may be encased in a cylindrical conduit (not shown) that extends between each support 40 and the lower housing 62. The upper housing 52 of the display assembly 50 may be attached to or integrated into the top surface 22 of the platform 20 so as to provide a convenient interface for user input and for display purposes. The lower housing 62 of the display assembly 50 may be attached to or integrated into the bottom surface 24 of the platform 20.

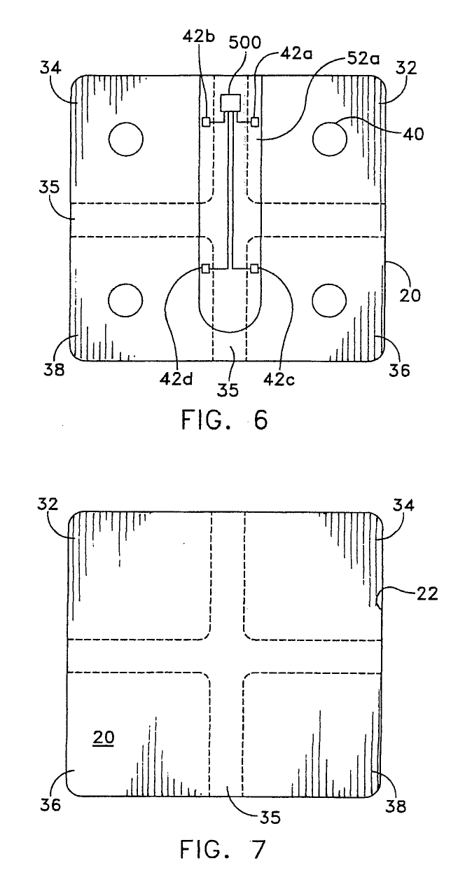

Referring again to FIG. 1A along with FIG. 7, visible light transmissive conductive electrodes (hereinafter in the following description, referred to simply as “transparent” for convenience) 32, 34, 36, and 38 are disposed on portions of the top surface 22 of the transparent platform 20. The transparent electrodes 32, 34, 36, 38 maybe composed, for example, of Indium Tin Oxide (ITO), zinc-doped indium oxide (IZO), and or other combinations of materials which enable visible light transmission therethrough. The transparent electrodes 32,34, 36, and 38 are electrically separated from one another by areas 35 of the platform top surface 22 not covered by the electrodes 32, 34, 36, and 38. The transparent electrodes 32, 34, 36, and 38 define respective quadrants A, B, C, and D. Electrodes 32 and 34 may be utilized for driving current through portions of the user's body and electrodes 36 and 38 may be utilized used for sensing a voltage potential between other portions of the user's body. The electrodes 32, 34, 36, and 38 may be formed by depositing a uniform, thin film of ITO over the entire top surface 22 of base 20, and then removng portions of the ITO to expose the areas 35 of the top surface 22 of the platform 20 between the electrodes 32, 34, 36, and 38. The thin film of ITO may be deposited using any conventional thin film deposition method, such as sputtering, and the portions of the ITO thin film may be removed using any conventional thin film removal method, such as wet etching. The ITO thin film may have a thickness which is less than 10,000 angstroms and typically between 1000 angstroms and 5000 angstroms. A feature of the ITO thin film is that the thickness is so small that the difference (i.e. height, environmental, and material characteristics) between the film and platform is imperceptible by the foot/body part of the user.

Typically, the aggregate area of the transparent electrodes 32, 34, 36, and 38 forms a majority of the area of the top surface 22 of the platform 20, and particularly, the areas that receive the feet of the user. Each of the transparent electrodes 32, 34, 36, and 38 is electrically coupled to a corresponding contact 42a, 42b, 42c, and 42d, which in the embodiment depicted in FIGS. 1A and 1B are disposed in or on the bottom surface 24 of the platform 20. The contacts 42a, 42b, 42c, and 42d communicate signal information to the body impedance and composition measuring circuitry 51 contained in the upper housing 52 of the display assembly 50, which measures body impedance and determines body composition. Each of the transparent electrodes 32, 34, 36, and 38 may be directly coupled to its corresponding contact 42a, 42b, 42c, and 42d in or on the bottom surface 24 of the platform 20 via an electrical conductor (not shown), or alternatively, may be indirectly coupled (e.g. capacitively coupled) to its corresponding contact 42a, 42b, 42c, 42d through the medium of the platform 20.

FIG. 1B, which is a partial cross section view of the front end of the body fat scale 10 of FIG. 1A, illustrates a method (e.g. resistive or capacitive) for electrically coupling each of the thin transparent electrodes 32, 34, 36, and 38 with their corresponding contacts 42a, 42b, 42c, and 42d through the medium (e.g. glass or plastic) of the platform 20. As shown, the transparent platform 20 has a thickness t and recesses 21 of depth x (where x is less than t) formed in the bottom surface 24 thereof, each of which accommodates one of the contacts 42a, 42b, 42c, and 42d. In the shown exemplary embodiment, each of the contacts 42a, 42b, 42c, and 42d is disposed on top of (or are integral with) a corresponding one of the sensor containing supports 40. The contacts 42a, 42b, 42c, and 42d may be electrically coupled via leads 43 to their respective conductors 66 for electrically connecting to the body impedance and composition measuring circuitry 51 contained within the upper housing portion 52 of the display assembly 50. An aperture 23 extending through the platform 20, enables lead connection between the lower housing 62 and the upper housing 52 of the display assembly 50. The lower housing 62 (which may be symmetrically shaped or complementary with the upper housing 52) may also house a power source, such as a battery (not shown) for powering the scale 10. The display assembly 50 may be located in a central portion of the platform 20. The upper housing 52 may further comprise a display portion 54 such as an LCD display, for example, for viewing the results of the weight and body composition measurements, and an interface portion 56 for entering user input data. Interface portion 56 may include push buttons sufficiently sized for enabling user data to be input into the body impedance and composition measuring circuitry 51, via a user's toe.

Referring again to FIG. 1A, in accordance with another aspect of the invention, the scale 10 may further include a body impedance/body fat activation and user selection switch 200 located at a central, lower portion of the scale platform 20. The selection switch 200 maybe implemented as a visible light transmissive electrode disposed on the top surface 22 of the light transmissive platform 20. The light transmissive electrode switch 200 may be composed, for example, of Indium Tin Oxide (ITO). The light transmissive electrode switch 200 is operatively coupled to the processor for activating the body impedance and body fat determination process. In an exemplary embodiment, user contact of the light transmissive electrode switch 200 at either a first position area (denoted by numeral 1 in FIG. 1A) or a second position area (denoted by numeral 2 in FIG. 1A) activates the body impedance circuitry, which causes a microprocessor of the circuitry to retrieve input characteristics of the particular user associated with the selected switch position areas 1 and 2. Although not shown, a second light transmissive electrode switch may be employed as an on/off switch connected to appropriate circuitry for activating/deactivating the scale 10.

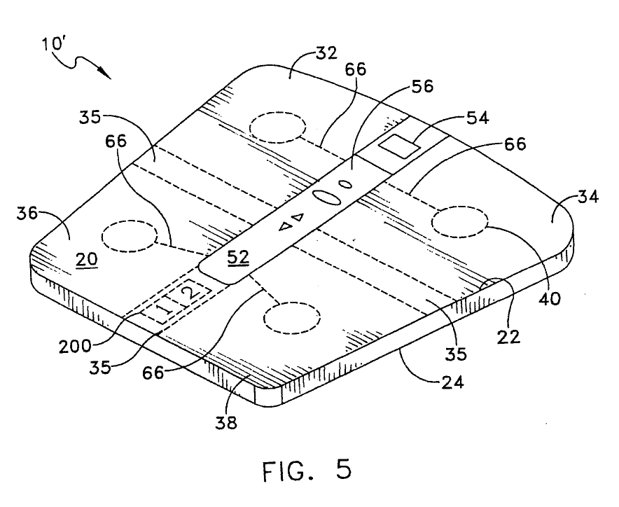

FIGS. 5 and 6 collectively show an alternative embodiment of the scale of the present invention, denoted by numeral 10′. FIG. 5 is a top, perspective view of the scale 10′ and FIG. 6 is a bottom plan view of the scale 10′ omitting the lower housing 62 of the display assembly 50, and the conductors 66, to more clearly illustrate the connectivity of each the contacts 42a, 42b, 42c, and 42d of this embodiment, as will not be explained. The scale 10′ is substantially identical to the scale 10 of FIG. IA, except that the contacts 42a, 42b, 42c, and 42d of scale 10′ are disposed at various locations on or in an inner surface 52a of the upper housing 52 of the display assembly 50. This arrangement allows the contacts 42a, 42b, 42c, and 42d to also rest on the top surface 22 of the platform 20, such that each of the contacts 42a, 42b, 42c, and 42d physically engages a predetermined portion of its respective transparent electrode 32, 34, 36, and 38 corresponding to quadrants A, B, C, and D, for providing electrical contact and communication with the circuitry 51 contained within the upper housing 52 of the display assembly 50 that determines body impedance and composition. In this arrangement, the contacts 42a, 42b, 42c, and 42d, due to their position on or in the inner surface 52a of the upper housing 52, are not visible to a user, nor are any of the conductive leads which extend from the contacts to the circuitry 51 contained within upper housing 52. As discussed earlier with respect to the embodiment of the scale 10 shown in FIG. lA, a transparent electrode on/off switch (not shown) may also be disposed on the top surface 22 of the platform 20 for activating/deactivating the scale 10′.

The body composition or body fat scale of the present invention may be assembled in accordance with the following an exemplary process. The process may be commenced by attaching each of the supports 40 to portions of the bottom surface 24 of the platform 20 such that the supports 40 are symmetrically oriented about the platform 20. The upper housing portion 52, which includes the processor and its associated circuitry for carrying out the functions of the body fat scale, and the contacts 42a, 42b, 42c, 42d, which may be disposed on its inner surface 52a, are coupled to the top surface 22 of the platform 20 such that each of the contacts 42a, 42b, 42c, and 42d is in electrical communication with its corresponding transparent, conductive electrode 32, 34, 36, and 38. An aperture 23 (FIG. 1B) may be formed through a center of the platform 20. The lower housing portion 52 is coupled to the bottom surface 24 of the platform 20 and the conductors 66 from the supports 40 are connected through the lower housing portion 52 to the body impedance and composition measuring circuitry 51 integrated in the upper housing portion 52, via the aperture 23 in the platform 20.

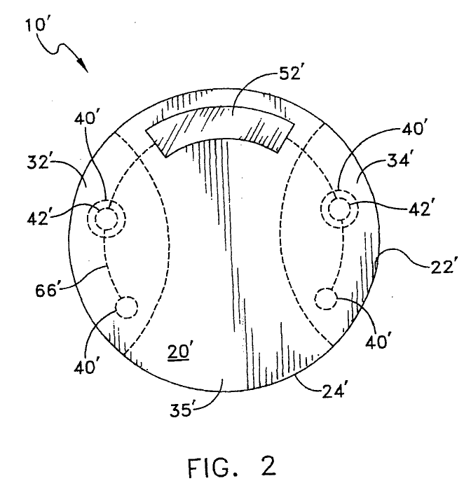

FIG. 2 shows another embodiment of the body composition or body fat scale of the present invention, denoted by numeral 10″. The scale 10″ comprises a round, transparent platform 20′ having a substantially planar top surface 22′ and a bottom surface 24′. A plurality of supports 40′ are disposed on the bottom surface 24′ of the platform 20. Electrical conductors 66′ may extend beneath the bottom surface 24 of the platform 20 for coupling each of the sensors contained with the supports 40 to body impedance and composition measuring circuitry integrated within a housing 52′ of a display assembly. The display assembly may be attached to the top surface 22′ or the bottom surface 24′ of the platform 20′ so as to provide a convenient interface for user input and for display purposes. Transparent (visible light transmissive), conductive electrodes 32′ and 34′ are disposed on the top surface 22′ of the platform 20′. These transparent conductive electrodes have a uniform thickness and a curved contour as shown in FIG. 2. One of the electrodes 32′ and 34′ operates to receive a foot of a user for applying a signal to the user's body and the other one of the electrodes 32′ and 34′ operates to receive the other foot of the user for sensing a resultant voltage signal therefrom for determining a body impedance. Contacts 42′ are disposed on the bottom surface 22′ of the platform 20′. The contacts 42′ operatively communicate the signal information to the body impedance and composition measuring circuitry, as previously discussed with respect to FIG. 1A. The transparent electrodes 32′ and 34′ are separated by area 35′ where no electrode material is coating the top surface 22′ or the platform 20′.

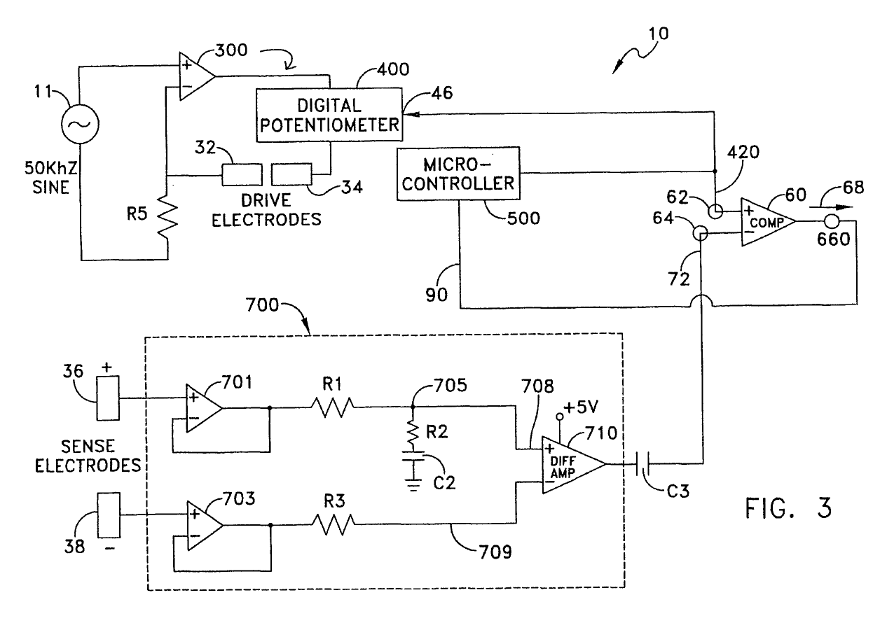

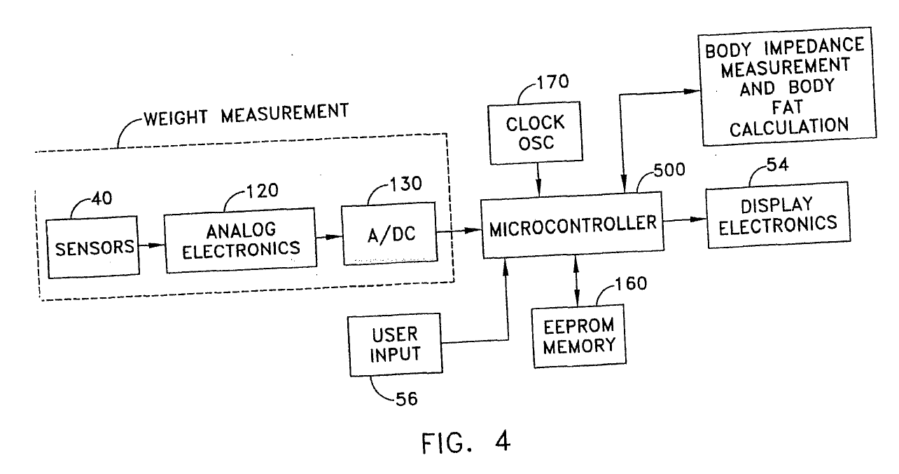

FIG. 3 is a schematic illustration of exemplary circuit for performing functions associated with the body fat scale of the present invention, and FIG. 4 is a block diagram of the major functional components of the body composition or body fat measuring scale of the present invention.

As shown in FIG. 3, a sine wave (steady state) voltage source 11 of about 50 kilohertz (kHz) is converted to a current drive source of less than 1 milli ampere (mA) at 50 kHz using a conventional voltage to current converter circuit 300. A pair of drive electrodes, which may be earlier described electrodes 32 and 34, interface with a first portion of the user's body (e.g. are in contact with the toes of the feet) and are in the feedback loop of amplifier 300. A digitally controlled potentiometer 400 is also in the feedback loop of amplifier 300. Accordingly, the same current that goes through the user's body goes through the potentiometer 400. The potentiometer 400 has a center tap 46 or wiper position that is selected digitally, via microcontroller 500. The center tap 46 of the potentiometer 400 is n times the voltage of the 50 kHz oscillator 11 (0<n<1) and is applied to an input terminal 62 of a comparator 60. The digital potentiometer 400 is stepped in increments of 10ohms so as to range between 0 and 1000 ohms (1K). Thus, step n ranges from 0 to 100 steps in 10 ohm increments.

A pair of sense electrodes, which may be earlier described electrodes 36 and 38, are used as voltage sense electrodes where no current flows. The electrodes 36 and 38 interface with a second portion of the user's body (e.g. are in contact with the heels of the feet). The potential across the electrodes 36 and 38 is applied as input to a standard 3 op-amp instrumentation amplifier (differential amplifier) arrangement 700. The amplifier arrangement 700 comprises first and second buffer amplifiers 701 and 703, each of the buffer amplifiers 701 and 703 having its non-inverting input terminal coupled to the electrodes 36 and 38, respectively. The outputs of each of the buffer amplifiers 701 and 703 pass through respective resistors R1 and R3 and are fed into terminals 708 (non-inverting input) and 709 (inverting input) of a differential amplifier 710. Resistor R2 and capacitor C2 are serially coupled between node 705 (non-inverting input) and ground potential and operate to filter noise components and to protect the bias point of the differential amplifier 710 (V/2). The output signal 72 of the differential amplifier arrangement 700 is applied to terminal 64 of the comparator 60 through a coupling capacitor C3. The comparator 60 accepts as an input at terminal 62 the voltage signal 420 developed at the center tap 46 of the digital potentiometer 400. The comparator 60 compares the microcontroller-selected voltage signal 420 developed at the tap position 46 of the digital potentiometer 400 with the voltage signal 72. The comparator 60 outputs a signal 68 at output terminal 660 based on the magnitude of the two input signals 420 and 72.

The comparator output 68 from output terminal 660 indicates whether the center tap 46 of the potentiometer 400 exceeds the voltage signal 72. The comparator 60 output is, in the preferred embodiment, a binary output signal corresponding to either a “high” (binary 1) or “low” (binary 0) state. The output signal 68 is applied via line 90 to the microcontroller 500. If signal 420 is greater than signal 72, the output signal 68 from the comparator 60 is “high”. In a preferred embodiment, this “high” signal indicates to the microcontroller 500 to provide a control signal to decrease the resistance at the center 46 of the digital potentiometer 400 so as to decrease the voltage of signal 420. The digital potentiometer 400 comprises n steps of a predetermined increment (for example 10 ohms). In this manner the resistance of the potentiometer 400 and hence voltage signal 420, is incrementally adjusted in response to the comparator output based on the step count n and the increment value. That is, the microcontroller 500, in response to output signal 68, sets or adjusts the center tap 46 of the digital potentiometer 400 to a different resistance value each iteration in order to find the point where the output of comparator 60 experiences a state transition. At the point where the voltage signal 420 equals (or is less than) voltage signal 72, the output of the comparator 60 transitions from a “high” to “low” value. When the output of the comparator 60 changes state (from “high” to “low”, for example) the comparator 60 is effectively nulled, and the microcontroller 500 in response to detection of a state change terminates further adjustment of the potentiometer resistor value. The bio impedance Z is then determined directly as the number of steps or adjustments n, times the value of the center tap resistance increment (e.g. 10 ohms) of the digital potentiometer 400. This corresponds to the voltage value of the sense electrodes 36 and 38 divided by the constant current at the drive electrodes 32 and 34.

Note that while the above description is predicated on a “high” to “low” transition of the comparator 60 and decrementing the voltage signal 420 applied at comparator terminal 62 through potentiometer adjustment, it is understood that a “low” to “high” transition detection and incrementing of the voltage signal 420 through adjustment of the potentiometer resistance is of course, also contemplated.

As shown in the functional block diagram of FIG. 4, a weight measurement is determined through the force sensors in the subassemblies or supports 40 comprising each of the load cells or other sensors within the platform to sense the weight of a user for generating a signal indicative of the relative amount of weight sensed. Analog electronics module 120 includes calibrating circuits, which enable each of the individual sensor elements to provide a response that reflects an accurate proportional share of the total weight applied to the platform, and a combining junction for combining each of the individually calibrated piezoresistive sensor signals. The analog electronics circuit is coupled to the sensors in supports 40 via conductors. Analog to digital converter 130 operates to convert the analog calibrated signal into a digital signal representation for input into processor (microcontroller) 500.

Processor 500 comprises a digital microprocessor controller having a clock oscillator 170 operating at approximately 4 MHz to generate a 50 KHz signal, memory 160 and user input interface for accepting data from a user. The digital microprocessor includes software programs or algorithms which operate to calculate body fat based on the determined body weight, the determined body resistance and patient data, such as height, age, and gender, for example.

It is to be understood that one skilled in the art may make many variations and modifications to that described herein. For example, the circuit for performing body fat measuring functions of the scale can also be embodied as a two wire system having a fixed value resistor and a fixed value capacitor forming an RC circuit and a body impedance which is connectable with the RC circuit. When a user stands on the scale, the RC time constants associated with charging and discharging of the fixed value capacitor can be measured and the corresponding body impedance determined. This and any other such variations are intended to be within the scope of the invention as defined in the appended claims.

What is claimed is:

1. An apparatus for measuring body composition, the apparatus comprising:
a rigid, light transmissive platform having a top surface for supporting a user standing on the apparatus, and a bottom surface;
a plurality of support assemblies associated with the bottom surface of the platform, for supporting the platform above a surface, at least one of the assemblies having a sensor for measuring a weight of the user; and
at least two, light transmissive conductive electrodes disposed over the top surface of the platform, the electrodes for contacting portions of the user's body; wherein the electrodes provide signal information about the user's body, which is used for measuring the composition of the user's body.

2. The apparatus of claim 1, wherein the signal information includes an alternating signal applied to the user's body by one of the at least two light transmissive electrodes and a signal outputted by the user's body and sensed by the other one of the at least two transparent electrodes.

3. The apparatus of claim 1, wherein the at least two light transmissive electrodes, in aggregate, occupy a majority area of the top surface of the platform.

4. The apparatus of claim 1, wherein the at least two light transmissive electrodes are electrically isolated from one another on the top surface of the platform.

5. The apparatus of claim 1, wherein the at least two light transmissive electrodes are each formed of Indium Tin Oxide.

6. The apparatus of claim 1, wherein the at least two light transmissive electrodes are uniformly disposed over the top surface of the platform and each define a quadrant.

7. the apparatus of claim 1, wherein each of the at least two light transmissive electrodes is coupled to a contact associated with a surface of the platform, the contacts for communicating signal information to a processor for measuring body impedance and determining body composition.

8. The apparatus of claim 1, wherein at least one of the at least two light transmissive electrodes is directly coupled to a contact associated with the bottom surface of the platform, the contact for communicating the signal information to a processor for measuring body impedance and determining body composition.

9. The apparatus of claim 1, further comprising a display assembly having a housing and at least one contact disposed on an inner surface of the housing of the display assembly, the at least one contact for communicating the signal information to a processor for measuring body impedance and determining body composition, the at one contact being directly coupled to at least one of the least two light transmissive electrodes.

10. The apparatus of claim 1, wherein at least one of the at least two light transmissive electrodes in indirectly coupled to a contact associated with the bottom surface of the platform, the contact for communicating the signal information to processor for measuring body impedance and determining body composition.

11. The apparatus of claim 10, wherein the indirect coupling of the at least one of the at least two light transmissive electrodes is through the platform.

12. The apparatus of claim 1, further comprising a display assembly for displaying a measured body weight of the user and/or a body composition of the user.

13. the apparatus of claim 1, further comprising a light transmissive electrode operable as a switch for activating or deactivating the apparatus, the light transmissive electrode disposed over the top surface of the platform.

14. An apparatus for analyzing human body composition based on a bioelectrical impedance method, the apparatus comprising:
a light transmissive platform having top and bottom surfaces;
a plurality of light transmissive electrodes disposed over the top surface of the platform for contacting an upper or toe portion of a right foot, a lower or heel portion of the right foot, an upper or toe portion of a left foot, and a lower or heel portion of the left foot;
contacts disposed over one of the top surface and bottom surface of the platform and in electrical communication with the light transmissive electrodes for providing signal communication to a processor for measuring body impedance.

15. An apparatus for analyzing human body composition based on a bioelectrical impedance method, the apparatus comprising:
a light transmissive platform having top and bottom surfaces;
a first light transmissive electrode disposed over a top surface of the platform for contacting a left foot of a user;
a second light transmissive electrode disposed over the top surface of the platform for contacting a right foot of the user;
first and second contacts associated with one of the top surface and bottom surface of the platform, and respectively in electrical communication with the first and second light transmissive electrodes, the contacts providing signal communication to a processor for measuring body impedance.

16. The apparatus of claim 15, further comprising support assemblies associated with the bottom surface of the platform, the support assemblies providing at least one sensor for weight calculation to determine the user's weight.

17. A circuit for measuring body impedance, the circuit comprising:
a voltage source;
a current source;
a first pair of light transmissive electrodes disposed over a top surface of a light transmissive platform, the first pair of electrodes for applying the current source to a user's body;
a second pair of light transmissive electrodes disposed over the top surface of the platform, the second pair of light transmissive electrodes for sensing a voltage outputted by the user's body; and
a processor for measuring body impedance and determining body composition based on the sensed voltage signal in response to the applied current source.

18. The circuit of claim 17, wherein the voltage source comprises a self determining frequency source.
